(19) United States
(12) Patent Application Publication
(10) Pub. No.: US 2003/0059950 A1
(43) Pub. Date: Mar. 27, 2003

(54) Method and apparatus for measuring ultra-trace amounts of arsenic, selenium and antimony

(76) Inventor: Josef B. Simeonsson, Durham, NC (US)

Correspondence Address: Gilbreth & Associates, P.C., P.O. Box 2428, Bellaire, TX, 77402-2428, US

(21) Appl. No.: 10/096,330

(22) Filed: Mar. 11, 2002

Related U.S. Application Data
(60) Provisional application No. 60/274,570, filed on Mar. 9, 2001.

(51) Int. Cl. G01N 33/00

(52) U.S. Cl. 436/182; 436/73; 436/181; 436/164; 436/172; 423/644; 422/82.05; 422/82.08; 422/83

(57) ABSTRACT

An analytical apparatus including a hydride generator coupled with a a laser-based detection device, with the generator suitable for generating hydrides from a sample, and the detection device positioned to receive hydrides from the hydride generator, and to provide information regarding quantity, identity, presence and/or species of the hydride. A method of processing a sample includes generating hydrides from the sample, and generating information regarding quantity, identity, presence and/or species of the hydride.

### RELATED APPLICATION DATA

The present application claims priority of U.S. Provisional Patent Application Serial No. 60/274,570, filed Mar. 9, 2001, which application is also incorporated herein by reference.

### BACKGROUND OF THE INVENTION

1. Field of the Invention

The present invention relates to detection, speciation, identification, and/or quantification of materials. In another aspect, the present invention relates to methods and apparatus for detection, speciation, identification, and/or quantification of materials. In even another aspect, the present invention relates to methods and apparatus for detection, speciation, identification, and/or quantification of samples containing metaloid elements. In still another aspect, the present invention relates to methods and apparatus for detection, speciation, identification, and/or quantification of samples containing arsenic, selenium, and/or antimony. In yet another aspect, the present invention relates to methods and apparatus for chemically speciating selected arsenic and selenium anion species. In even still another aspect, the present invention relates to apparatus combining laser-induced fluorescence or laser-enhanced ionization with continuous flow-hydride generation for the detection, identification, and/or quantification of samples.

2. Related Art

There continues to be interest in determination, quantification and/or detection of samples. For example, the trace determination of arsenic (As), selenium (Se) and antimony (Sb) species because of concerns over pollution of the environment and the important roles these elements have in biological systems. In many instances the toxicity and/or essentiality of certain chemicals depends not only on their concentrations, but also on their chemical forms (i.e., “speciation”).

Hydride generation is a sample introduction technique that is useful for enhancing sensitivity and providing chemical speciation information, especially for metalloid elements. In addition, hydride generation provides the important capability of separating analyte species from the sample matrix.

Various hydride generation approaches have been combined with many atomic spectrometric techniques, including inductively coupled plasma atomic emission spectrometry (ICP-AES), inductively coupled plasma mass spectrometry (ICP-MS), atomic absorption spectrometry (AAS), atomic emission spectrometry (AES) and atomic fluorescence spectrometry (AFS).

For example, U.S. Pat. No. 5,250,260, issued Oct. 5, 1993, to Nakano et al., discloses a tape for detecting hydride the presence of hydride gases by taking advantage of coloring reactions whose light sensitivity is lowered to a minimum without impairing the inherent mechanical strength of the reagent holder. The detection tape is prepared by impregnating a tape of porous cellulose fibers containing a gas adsorbent such as a powder of silica and a moisture keeper of glycerin with a coloring reagent of silver perchlorate or silver para-toluenesulfonate and a light resistance enhancer of para-toluenesulfonic acid. When the detection tape is exposed to a hydride gas contained in the sampled gas, silver perchlorate or silver para-toluenesulfonate is reduced by the hydride gas to form a colloid of silver that remains as a trace of reaction. As the amount of the colloid of silver is proportional to the concentration of the gas, the concentration of hydride gas can be determined by measuring the optical density of the trace of reaction.

U.S. Pat. No. 6,083,758, issued Jul. 4, 2000, to Imperiali et al., discloses methods for screening peptides for metal coordinating properties and fluorescent chemosensors derived therefrom. These methods utilize chemosensors comprising polypeptides which coordinate to select metals and methods for selectively detecting the presence of metals.

U.S. Pat. No. 6,171,552, issued Jan. 9, 2001, to Takeya et al., discloses a hydride formation analytical apparatus which forms hydrides of target components contained in a sample liquid and then analyzes them. The hydride formation analytical apparatus comprises a sample-introducing part, a reagent-introducing part, a reaction part, a gas-liquid separating part and a detecting part, wherein an acid-feeding part and a reducing agent-feeding part are part of the reagent-introducing part; the hydride gas of the sample is formed by the aid of the acid and the reducing agent fed into the above reaction part; and this is introduced into the detecting part for analysis.

However, while these techniques provide a certain level of sensitivity, such may not be adequate at low concentration levels. For example, low concentrations of As, Se and Sb found in many environmental and biological samples often demand even higher levels of sensitivity than is provided by the above described techniques.

Thus, there is a need in the art for methods and apparatus for detection, speciation, identification, and/or quantification of materials.

There is another need in the art for methods and apparatus for detection, speciation, identification, and/or quantification of materials not suffering from the limitations or disadvantages of the prior art.

There is even another need in the art for methods and apparatus for detection, speciation, identification, and/or quantification of low concentrations of materials.

There is still another need in the art for methods and apparatus for detection, speciation, identification, and/or quantification of low concentrations of arsenic (As), selenium (Se) and antimony (Sb).

These and other needs of the present invention will become apparent to those of skill in the art upon review of this specification.

### SUMMARY OF THE INVENTION

It is an object of the present invention to provide for methods and apparatus for detection, speciation, identification, and/or quantification of materials.

It is another object of the present invention to provide for detection, speciation, identification, and/or quantification of materials not suffering from the limitations or disadvantages of the prior art.

It is even another object of the present invention to provide for detection, speciation, identification, and/or quantification of low concentrations of materials.

There is still another need in the art for methods and apparatus for detection, speciation, identification, and/or quantification of low concentrations of arsenic (As), selenium (Se) and antimony (Sb).

These and other objects of the present invention will become apparent to those of skill in the art upon review of this specification.

According to one embodiment of the present invention, there is provided an apparatus for processing a sample comprising a compound of interest. The apparatus includes a hydride generator suitable for generating a hydride of the compound from the sample. The apparatus further includes a laser-based detection device positioned to receive the hydride from the hydride generator, and to provide information regarding quantity, identity, presence and/or species of the hydride.

According to another embodiment of the present invention, there is provided an apparatus for processing a sample comprising a compound of interest. The apparatus includes a hydride generator suitable for generating a hydride of the compound from the sample. The apparatus further includes an electrothermal atomizer positioned to receive and atomize the hydride from the hydride generator. And, the apparatus includes a laser-based detection device positioned to receive the atomized hydride from the atomizer, and to provide information regarding quantity, identity, presence and/or species of the hydride.

In the above described apparatus, any suitable laser-based detection device may be utilized, but preferably a laser-induced fluorescence device or a laser-enhanced ionization device is utilized.

In the above described apparatus, any suitable hydride generator may be utilized, but preferably, a continuous flow hydride generator is utilized.

The above apparatus are suitable for processing any type of compounds, more preferably for processing metalloid compounds, most preferably a compound comprising at least one selected from the group consisting of arsenic, selenium and antimony.

According to another embodiment of the present invention, there is provided a method for processing a sample. The method includes the step of generating a hydride from the sample, and then generating information regarding quantity, identity, presence and/or species of the hydride.

According to even another embodiment of the present invention, there is provided a method for processing a sample. The method includes generating a hydride from the sample, atomizing the hydride and generating information regarding quantity, identity, presence and/or species of the hydride.

For the above described methods, the hydrides are generated utilizing a continuous flow hydride generator.

For the above described methods, generating information regarding quantity, identity, presence and/or species of the hydride, is carried out utilizing a laser-based system, preferably a laser-induced fluorescence device or laser-enhanced ionization device.

The above methods are suitable for any type of compounds, more preferably for processing metalloid compounds, most preferably a compound comprising at least one selected from the group consisting of arsenic, selenium and antimony.

These and other embodiments of the present invention will become apparent to those of skill in the art upon review of this specification.

### BRIEF DESCRIPTION OF THE DRAWINGS

FIG. 1 is a schematic representation of the LIF apparatus utilized in the Examples, showing excimer laser 201, dye laser 203, BBO frequency doubling crystal 205, Raman shifter 207, Pellin-Broca prism 208, beam stopper 209, argon feed 211, modified ICP torch 212, hydride feed 214, converging lens 216, monochromator 217, PMT 220, boxcar averager 223, and computer 224.

FIG. 2 is a schematic representation of the LIF apparatus utilized in the Examples, showing excimer laser 301, dye laser 303, frequency doubler 305, Raman shifter 307, Pellin-Broca prism 308, beam stopper 309, argon feed 311, modified ICP torch 312, hydride feed 314, boxcar averager 323, computer 224., amplifier 335, high voltage power supply 338, dye laser 331.

FIG. 3 is a schematic representation of the ETA-LIF apparatus utilized in the Examples, showing furnace controller 401, beam stopper 402, quartz tip 403, mirrors 404, laser beam 405, aperture 408, lens 410, monochromator 412, PMT 413, boxcar averager 414, computer 418, hydride feed 419, teflon tube 420, and graphite tube 422.

FIG. 4 is a schematic representation of the HG apparatus utilized in the present invention, showing acidified sample feed 501, pre-reduction coil 503, reaction coil 505, helium 506, dry air circulation 508, stream 509 to flame atomizer, Nafion tube dryer 510, teflon filter 512, gas liquid separator 513, mixing block 515, waste stream 518, pump 519, sodium borohydride feed 520.

FIG. 5 is a plot of Example data, showing the effect of the acid (HBr/HCl) concentration on the S/N for HG measurements of Se (triangles, HG-LEI), As (circles, HG-LIF) and Sb (squares, HG-LIF).

FIG. 6 is a plot of Example data, showing the effect of the NaBH₄ concentration on the S/N for HG measurements of Se (circles, HG-LEI), As (triangles, HG-LIF) and Sb (squares, HG-LIF).

### DETAILED DESCRIPTION OF THE INVENTION

The apparatus of the present invention includes a hydride generator and further includes a laser-based detection system.

It should be understood, that any suitable hydride generator may be incorporated into the apparatus of the present invention. Preferably, the apparatus of the present invention will comprise a continuous flow hydride generator. While it is believed that the apparatus of the present invention may be utilized for the detection, speciation, identification, and/or quantification of any chemical compounds, it is especially suitable for the detection, speciation, identification, and/or quantification of metalloids, especially arsenic, selenium and antimony. Thus, more preferably the hydride generator is suitable for generating hydrides of the metalloids, especially of arsenic, selenium and antimony.

It should be understood, that while any suitable laser-based detection device may be incorporated into the apparatus of the present invention, preferably the laser-based detection device is selected from the group consisting of laser-induced fluorescence (“LIF”) and laser-enhanced ionization (“LEI”) spectrometry devices.

In an alternative embodiment, the apparatus of the present invention may also include an atomizer, preferably, an electrothermal atomizer, utilized for in situ trapping and atomizing of the hydride generated by the hydride generator. More preferably an Ar shielded H₂—Ar flame atomizer.

The apparatus is also described in article by Pacquette et al. (including the present inventor Simeonsson), J. Anal. At. Spectrom., 2001, 16( ), 152-158, entitled, “A comparison of continuous flow hydride generation laser-induced fluorescence and laser-enhanced ionization spectrometry approaches for parts per trillion level measurements of arsenic, selenium, and antimony,” which article, and the 41 references cited therein, are incorporated herein by reference.

The method of the present invention for processing a sample includes generating a hydride and then obtaining information regarding quantity, identity, presence and/or species of the hydride.

In an alternative embodiment, the generated hydride is atomized prior to obtaining information regarding quantity, identity, presence and/or species of the hydride. Preferably, an electrothermal atomizer is utilized for in situ trapping and atomizing of the hydride.

While the method of the present invention may be carried out utilizing any suitable apparatus, it is preferably carried out using the above described apparatus.

The present invention will find utility in any type of field where it is necessary to process a sample to detect, identify, speciate and/or quantify materials. More specifically, the present invention will find utility where it is necessary to process a sample to detect, identify, speciate and/or quantify samples containing at least one of arsenic, antimony, and selenium.

As a non-limiting example, the present invention will find utility in environmental, medical/clinical, and/or industrial, manufacturing, research, academic fields, in situations where it is desired to process a sample to detect, identify, speciate and/or quantify. Potential uses of the present invention in these fields may include routine measurement applications in environmental monitoring (air, food and water analyses), medical/clinical screening, and industrial quality control.

### EXAMPLES

The following examples are provided merely to illustrate some of the embodiments of the present invention, and are not meant to and do not limit the scope of the claims of the invention.

### Laser Systems

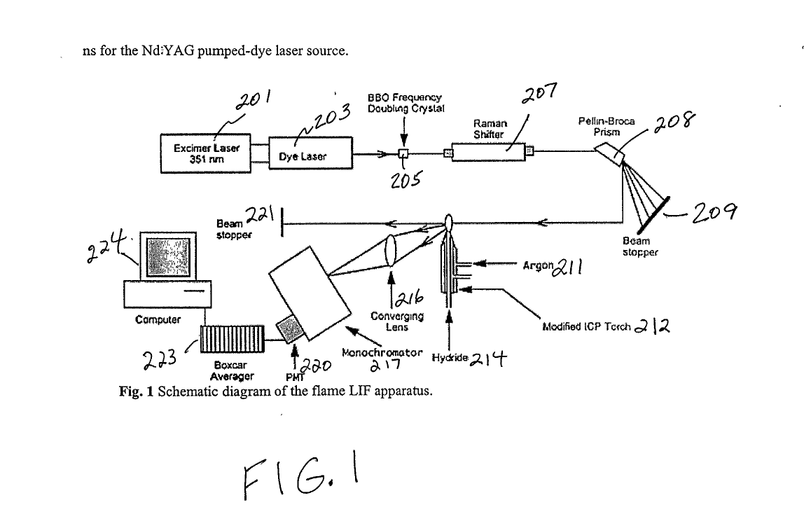

The LIF apparatus utilized in the these Examples, consists of a XeF excimer laser (LPX 210i, Lambda Physik, Ft. Lauderdale, Fla.) operating at 351 nm and a pulse width of 30 ns, which served as a pump source for a dye laser (Scanmate 2, Lambda Physik). FIG. 1 is a schematic representation of this LIF apparatus, showing excimer laser 201, dye laser 203, BBO frequency doubling crystal 205, Raman shifter 207, Pellin-Broca prism 208, beam stopper 209, argon feed 211, modified ICP torch 212, hydride feed 214, converging lens 216, monochromator 217, PMT 220, boxcar averager 223, and computer 224.

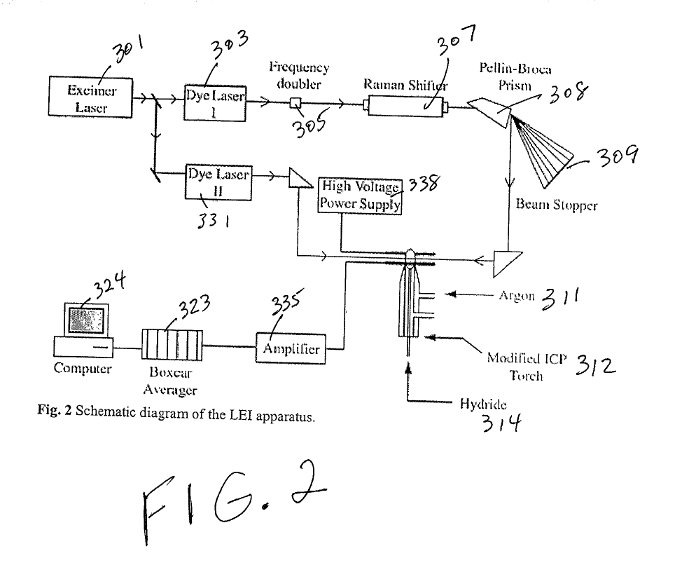

The LEI apparatus utilized in these Examples, consists of a Nd:YAG laser (Surelite II-10,Continuum, Santa Clara, Calif., pulsewidth of 7-8 ns) operating at a repetition rate of 10 Hz, which served as the pump source for another dye laser (Scanmate 2, Lambda Physik). FIG. 2 is a schematic representation of this LEI apparatus, showing excimer laser 301, dye laser 303, frequency doubler 305, Raman shifter 307, Pellin-Broca prism 308, beam stopper 309, argon feed 311, modified ICP torch 312, hydride feed 314, boxcar averager 323, computer 224., amplifier 335, high voltage power supply 338, dye laser 331.

The dye lasers of both laser systems produced radiation near 230-235 nm by frequency doubling of 460-470 radiation using Coumarin 460 dye, (Exciton, Dayton, Ohio) in a BBO I crystal. Frequency doubling typically reduced the pulse widths to 18 ns for the excimer pumped dye lasers. Tunable radiation from 193-197 nm was then produced by stimulated Raman scattering (SRS) of the 230-235 nm radiation in a 0.3 m Raman cell (Light Age Inc., Somerset, N.J.) filled with 100 psi H2. The desired outputs from the SRS cell were separated from other wavelengths using a quartz pellin broca prism and directed to the atomizer. Maximum energies achieved at 193-197 nm were 8-10 μJ for XeF excimer and Nd:YAG pumping, respectively. The pulse widths of the SRS pulses were approximately 8-10 ns for excimer pumped-dye laser sources and 4-5 ns for the Nd:YAG pumped-dye laser source.

For the LEI measurements, two dye lasers (Scanmate 2 and Fl 3001, Lambda Physik) were simultaneously pumped by the XeF excimer operating at 30 Hz.¹ In this two step excitation LEI laser system, the Scanmate 2 produced far UV radiation as described above, while the second dye laser produced radiation near 433-436 nm by using Coumarin 440 dye (Exciton). The spatial profile of the laser beam was slightly elliptical and typically had a diameter (major axis) of 3-4 mm at the flame and ETA atomizers in all Examples.

### Flame LIF System

A hydrogen flame from a quartz tube (¼ inch. i.d.) and a H2/Ar flame from a modified inductively coupled plasma (ICP) torch (¼ inch i.d.) were used as the atomizers for the LIF. The former burner did not utilize an exterior sheath of flowing Ar and did not provide as high a sensitivity as the modified ICP torch burner. This burner was only used for initial characterization Examples, and included the speciation Examples. All of studies were conducted using the shielded flame including the sensitivity and LOD determinations for the LIF and LEI techniques.

The laser beam intersected the flame at a height of 2-3 mm above the tip of the quartz tube and was adjusted to optimize the signal for each element (FIG. 1). The fluorescence emission of the elements was collected at 90° to the laser beam direction. The probe region of the flame atomizer was imaged with unit magnification onto a 0.15 m monochromator (1000 μm vertical slits, f/4, Spectra Pro 150, Acton Research, Acton, Mass.). Fluorescence radiation was detected using a photomultiplier tube (PMT) (R166UH, Hamamatsu, Bridgewater, N.J.). Neutral density filters were used to attenuate the fluorescence emission at high signal levels. The anode output of the PMT was sampled by a boxcar averager (SR250, Stanford Research Systems, Sunnyvale, Calif.), which was connected to an analog to digital converter/computer interface for acquisition by a personal computer. The boxcar gate widths used in these studies ranged from 10 ns for the ETA-LIF measurements to 20 ns for the flame LIF measurements.

### ETA-LIF System

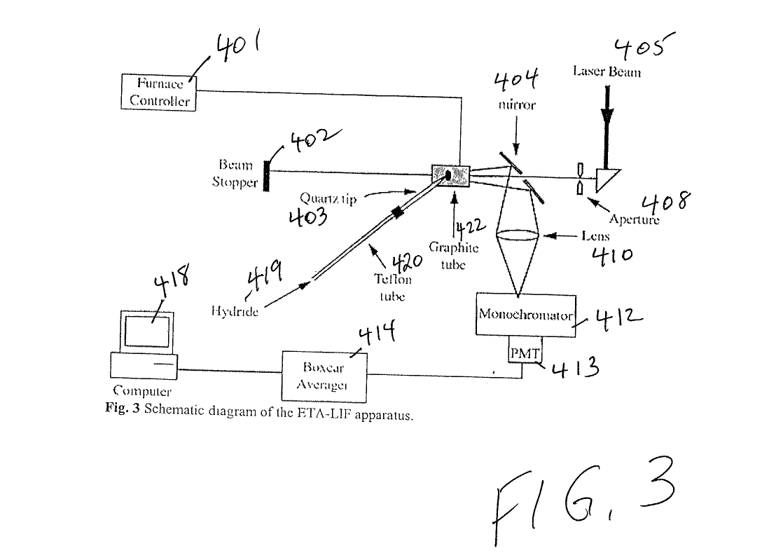

For the ETA-LIF system utilized in these Examples, the windows at the entrance and exit of the furnace housing were mounted at an angle of 56° to the laser beam to reduce laser scattering. Fluorescence was collected by a pierced mirror (50 mm diameter) set at a 45° angle along the incident laser beam axis and imaged 1:1 by a 2-inch diameter lens onto the entrance slit of a monochromator (spectra Pro-275, f/3.5. Acton Research Corp.) and detected using a solar blind PMT tube. FIG. 3 is a schematic representation of the ETA-LIF apparatus utilized in the Examples, showing furnace controller 401, beam stopper 402, quartz tip 403, mirrors 404, laser beam 405, aperture 408, lens 410, monochromator 412, PMT 413, boxcar averager 414, computer 418, hydride feed 419, teflon tube 420, and graphite tube 422.

The signal acquisition system (including the boxcar, A/D converter and personal computer) was identical to that described previously for the flame HG-LIF systems. The graphite furnace (HGA-400, Perkin-Elmer, Norwalk, Conn.) in this system is utilized both as the trapping cell and atomizer. Trapping and atomization took place in a 3 cm long graphite tube (0.5 cm i.d.) fitted with a L'vov platform.

### LEI System

In these Examples, analyte ionization was detected using two water-cooled electrodes in a flame that was irradiated by two collinear and counter propagating laser beams (Scanmate 2 and Fl 3001, Lambda Physik) (FIG. 2). The temporal overlap of the two laser pulses was ensured by delaying the first (earlier) beam with an optical delay line and measuring their arrival with a fast photodiode (DET200, Thorlabs, Inc., Newton, N.J.) at the flame atomizer. The flow rates of the flame gases were varied to optimize the LEI signal. The mixing ratios were different from those used for the LIF measurements, which was expected due to the role of thermal ionizations in the flame atomizer during LEI.

The cathode was biased at −1000 V voltage with a power supply (247, Keithley, Cleveland, Ohio) and the signal was measured at the anode via a capacitively coupled high bandwidth transimpedance amplifier (Al, Thorn-EMI, Rockaway, N.J.), whose output was connected to a boxcar averager (Stanford Research Systems). Although the amplifer bandwidth could accommodate response times as short as 20 ns, signal to noise studies performed in the laboratory indicated that a response time of 2 ms was optimal for this system.

### HG Apparatus

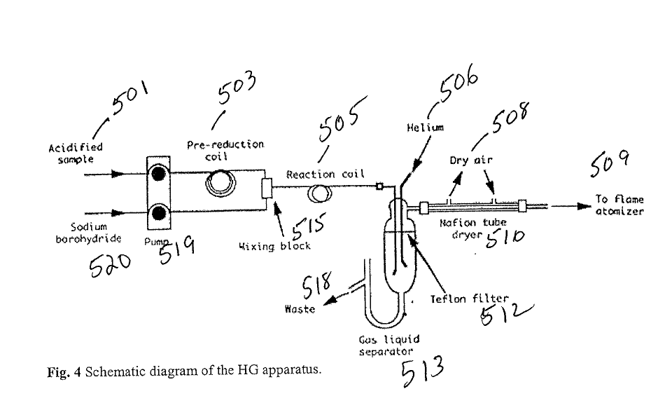

The HG system used in these Examples is a continuous flow system and consists of a four channel peristaltic pump (Model RP-1, Rainin Instrument, Co. Inc. Emeryville, Calif.) with tygon tubing, a gas liquid separator made of pyrex glass, a nafion tube dryer (MD series, Permapure Inc., Toms River, N.J.) and reaction coils of various lengths (1-2 m, 1 mm i.d.). FIG. 4 is a schematic representation of the HG apparatus utilized in the present invention, showing acidified sample feed 501, pre-reduction coil 503, reaction coil 505, helium 506, dry air circulation 508, stream 509 to flame atomizer, Nafion tube dryer 510, teflon filter 512, gas liquid separator 513, mixing block 515, waste stream 518, pump 519, sodium borohydride feed 520.

A prereduction coil of about 10 m was used for Se speciation. All connections to the HG apparatus were made with tygon tubing, teflon swagelok connectors and teflon tubing (¼ inch o.d., Swagelok, Co. Solon, Ohio). The flow rates of the NaBH4 and sample were controlled by using tubing with different diameters. The flow rates of all the gases and solutions used are shown in Table 1.

TABLE 1
Operating conditions for the HG system

| | HG-LIF/ | HG-LEI/ | HG-ETA-LIF/ |
|---|---|---|---|
| | ml min⁻¹ | ml min⁻¹ | ml min⁻¹ |
| Sample flow rate | 10 | 10 | 4 |
| NaBH₄ flow rate | 5.4 | 5.4 | 2 |
| He flow rate | 90 | 90 | 90 |
| Air flow rate (Nafion tube) | 1140 | 1140 | 1140 |
| Ar flow rate (flame) | 1277 | 1240 | None |
| H₂ flow rate (flame) | 162 | 545 | None |

### Reagents and Standards

Stock solutions of 1000 ppm of, Se (Perkin-Elmer), As (Fisher Scientific) and Sb (Perkin-Elmer) were used to prepare calibration standards by serial dilutions in 31% (v/v) concentrated HBr (Aldrich, Milwaukee, Wis.), 3.5% (v/v) concentrated HCl (TraceMetal, Fisher Scientific) and 10% (v/v) concentrated HCl (Fisher Scientific), for Se, As and Sb, respectively. All acid solutions were made by using 18 MW cm deionized water, which was obtained from an E-pure system (Barnstead/Thermolyne, Dubuque, Iowa). Additional stock solutions of 1000 ppm of As (III) or As (V) were prepared by dissolving As203 (purity 99.95-100.05%, Alfa Aesar, Johnson Matthey, Ward Hill, Mass.) or As205 (purity 99.9%, Alfa Aesar) in 5 M HCl and diluted with deionized water. Stock solutions (1000 ppm) of Se(IV) or Se (VI) were prepared by dissolution of Na2SeO3 (purity 99.75%, Alfa Aesar) or Na2SeO4. 10H2O (purity 99.9% Alfa Aesar) in deionized water. The pH values of the As sample solutions were adjusted with concentrated HCl and Tris-HCl buffer (ultra pure grade, Amresco, Solon, Ohio).

Solutions of NaBH4 (Aldrich, Milwaukee, Wis.) were prepared by dissolving NaBH4 powder in deionized water, and stabilized with 0.4% (m/v) NaOH pellets (Fisher Scientific). The Ir matrix modifier solution was prepared by diluting a commercial atomic absorption modifier solution (1000 mg/L Ir) (Perkin Elmer) in deionized water. The Pd matrix modifier was also prepared in deionized water by diluting a commercial absorption modifier solution (10,000 mg/L Pd as Pd(NO3)2, Perkin-Elmer).

### Procedure

### Determination of Se, As and Sb

As one of the objects of the present invention is the determination of ultratrace levels of As and Se, only these two elements were studied by all three approaches. Studies of Sb were performed using the flame HG-LIF approach only. Flow rates of the solution and gases used for HG in these studies are given in Table 1. For the determination of Se, an acidified sample containing 31% HBr5, and a 0.7% NaBH4 solution were pumped using two channels of a peristaltic pump at a flow rate of 10 ml/min and 5.4 ml/min, respectively. The generation of the H2Se and excess H2 produced in the reaction coil were carried from the gas liquid separator in a flow of He (90 ml/min) through the nafion tube dryer to the atomizer.5,20 A teflon filter (25 mm diameter) was placed in the gas liquid separator to reduce the amount of liquid aerosol transported to the dryer and atomizer. Measurements were taken at least 1 min. after beginning the sample and NaBH4 to allow equilibration and stabilization of the hydride signals. Determinations of As and Sb were carried out in a similar way, except that 1.5% NaBH4 and a 3.5% concentrated HCl sample solution were used for As, while 1% NaBH4 and 10% concentrated HCl sample solution were used for Sb.

In the HG-ETA-LIF studies, the ETA was used both for trapping of the hydride and atomization of the trapped species. The hydride trapping process requires initial conditioning of the graphite tube (which was fitted with a L'vov platform) prior to taking measurements.²,17 HG of As and Se was performed according to the above procedure. For As measurements the tube was modified only once by transferring 3×40 μl aliquots of 1000 mg l-1 solution of Ir matrix modifier by a micro-pipette onto the L'vov platform. This was followed by a conditioning step, according to the program in Table 2, to facilitate the trapping of the hydrides in the preheated graphite furnace. During HG, the hydride was delivered by a quartz tube inserted in the dosing hole of the graphite tube so that it just rested on the L'vov platform. After trapping (30 s) of the hydride, the quartz tube was removed and atomization was performed using the program outlined in Table 2. Trapping and atomization of Se was accomplished by conditioning of the graphite tube with one 20 ml aliquot of Pd matrix modifier prior to each measurement. After the Pd solution was dried, the hydride was trapped and atomized according to the program given in Table 3.

TABLE 2
Furnace temperature program for conditioning, hydride trapping
and atomization for HG-ETA-LIF measurements of As

| | | | | | Argon/ |
|---|---|---|---|---|---|
| | Step | Temperature/° C. | Ramp/s | Hold/s | ml min⁻¹ |
| Iridium | 1 | 110 | 25 | 40 | 300 |
| conditioning | 2 | 130 | 20 | 30 | 300 |
| | 3 | 1200 | 20 | 50 | 300 |
| | 4 | 2000 | 1 | 5 | 0 |
| Hydride | 1 | 250 | 1 | 30 | 0 |
| trapping | | | | | |
| Atomization | 1 | 20 | 1 | 15 | 300 |
| | 2 | 250 | 1 | 15 | 0 |
| | 3 | 2000 | 0 | 5 | 0 |
| | 4 | 2100 | 1 | 3 | 300 |

TABLE 3
Furnace temperature program for conditioning, hydride trapping
and atomization for HG-ETA-LIF measurements of Se

| | | | | | Argon/ |
|---|---|---|---|---|---|
| | Step | Temperature/° C. | Ramp/s | Hold/s | ml min⁻¹ |
| Palladium | 1 | 120 | 5 | 25 | 300 |
| conditioning | 2 | 400 | 5 | 15 | 300 |
| Hydride | 1 | 200 | 1 | 120 | 0 |
| trapping | | | | | |
| Atomization | 1 | 400 | 5 | 5 | 300 |
| | 2 | 2200 | 0 | 3 | 0 |
| | 3 | 2650 | 1 | 1 | 300 |

### Speciation of Se and As

For the selective speciation of Se(IV)/Se(VI), 65% HBr and NaBH4 were again used as the acid and reducing agent. In speciation, the use of HBr is important since it has better reductive selectivity for Se(VI) than HCl.5 In addition, the bromine formed does not cause back oxidation of Se(IV) to Se(VI), as does the chlorine formed when HCl is used. Acidified samples were made of equal concentrations of Se(IV)/Se(VI) ranging from 0.05 ng ml-1 to 0.7 ng ml-1. Each sample was prereduced in a coil (10 m) immersed in a beaker of boiling water (FIG. 4). The sample was further reduced with NaBH4 in the mixing block and reaction coil (1.5 m) and then cooled by immersing the latter in a beaker containing ice water to cool the sample. The measurements were repeated using identical concentrations of sample solutions, but without prereduction of the sample. A total of 4-5 minutes was required for prereduction and HG.

Both As (III) and (V) are reduced at pH=0, while only As (III) is reduced at pH=6. For the selective determination of As(III)/As(V), one set of samples containing equal concentrations of both oxidation states ranging from 0.05 ng ml-1 to 1 ng ml-1 was prepared in 4% concentrated HCl (pH<1) and another set of samples was prepared in a 2.5 M Tris-HCl buffer solution (pH=6). The samples in the HCl solution were reduced using 3% NaBH4 in a reaction coil (1.5 m). Comparison measurements of the identical samples in Tris-HCl buffer were also performed. Changes between concentrations required 2-3 minute for the signals to stabilize and be measured.

### Optimization of HBr, HCl and NaBH4 Concentrations

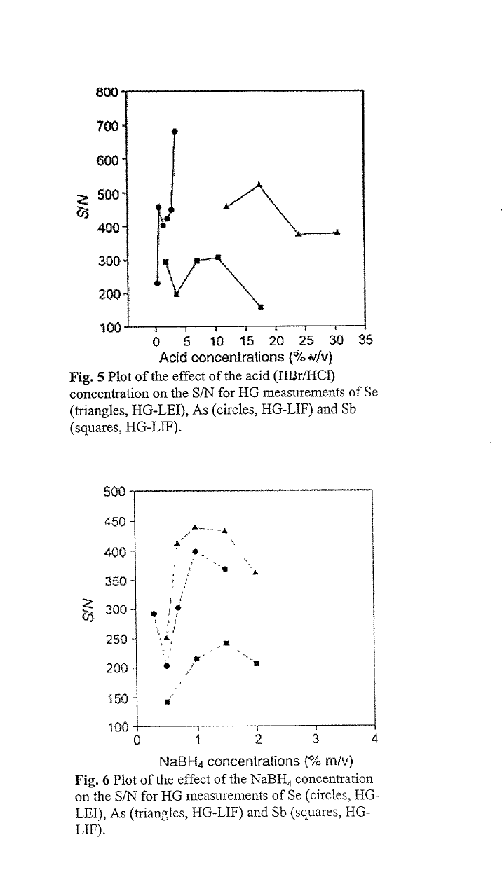

To determine the optimum HBr concentration for Se, different concentrations in the range of 12-31% (v/v) were used while keeping all other parameters constant. FIG. 5 shows that the optimum concentration of HBr for the production of H2Se from Se(IV) solutions was found to be 18%. This concentration would normally have been used, however, in these studies a higher concentration was used to ensure the complete reduction of Se(VI) to Se(IV) during speciation measurements. FIG. 5 also shows that the optimal concentrations of HCl for As and Se were 3.5 and 10% (v/v), respectively. These concentrations were used during the Examples. The optimum NaBH4 concentrations for As, Se and Sb were also obtained by independently varying the concentrations of NaBH4 from 0.2-1.5% (m/v), while keeping all other parameters constant (FIG. 6). For As and Sb, the general trend was a gradual increase in S/N up to concentrations of NaBH4 to 1 and 1.5%, and then a gradual decrease. A sharp increase in S/N was observed for Se at 1% NaBH4 concentration. The plots indicate that the optimal concentrations were 1% NaBH4 for As and Se, and 1.5% NaBH4 for Sb.

### Results and Discussion

### HG Flame LIF of As, Se and Sb

Laser induced fluorescence (LIF) measurements of As were accomplished using laser excitation at 197.197 nm from the 4p3 4So3/2 ground state to the 4p2 5s 4P1/2 excited state at 50694 cm-1, with fluorescence detection at 249.291 nm from the 4p2 5s 4P1/2 excited state to the 4p3 2Do 3/2 state at 10592 cm-l. Measurements of Se were performed using excitation at 196.026 nm from the 4p4 3P2 ground state to the 4p35s 3So1 excited state at 50996 cm-1, with fluorescence detection at 203.985 nm from the 4p35s 3So1 to the 4p4 3P1 state at 1989 cm-1. Similarly, LIF detection of Sb was accomplished using laser excitation at 231.147 nm from the 5p3 4So3/2 ground state to the 6s 4P1/2 state at 43249 cm-1 and fluorescence emission was measured at 287.792 cm-1 from the 6s 4P1/2 state to the 5p3 4Do3/2 state.

The best results were obtained using a H2 flame shrouded by flowing Ar gas as the LIF atomizer. This flame is a relatively quiet atomizer that has a low fluorescence background, and its combination with HG sample introduction and LIF detection provided excellent sensitivities for all three elements. It is noteworthy that the high sensitivity achieved in this system allowed concentrations as low as 3 pg ml-1 to be measured routinely. Excellent linearity was observed for all three HG-LIF approaches and the linear dynamic range extended over at least 3 orders of magnitude from the limit of detection (LOD) to 10 ng ml-1. Higher concentrations were not measured due to concerns about potential contamination of the apparatus. Due to a small background signal observed for each element (believed to be due to the reagents), the blanks were measured with the laser detuned away from the analyte wavelength. Limits of detection (3s for 16 blank measurements) of 0.3 pg ml-1, 0.09 pg ml-1 and 0.2 pg ml-1 were obtained for As, Se and Sb, respectively (Table 4). It is noteworthy that the LODs obtained in these studies using HG-LIF are equal to (for As) or lower than (for Se and Sb) the lowest results reported by any measurement approach, including HG-ICP-MS techniques.

TABLE 4
LODs for several continuous flow HG-atomic spectrometry techniques

| | | |
|---|---|---|
| | | LOD/pg ml⁻¹ |
| | | As |
| | Se | |
| | Sb | |
| HG-LIF^{a} | | 0.3 (2)^{b}0.09 (0.5)^{b}0.2 (1)^{b} |
| (flame) | | |
| HG-LEI^{a} | | 50 (100)^{b}2 (6)^{b} |
| (flame) | | |
| HG-ICP-MS | | 7⁹0.5⁹2⁹ |
| | | 0.3¹¹40¹⁰ |
| | | 20⁸20⁸ |
| HG-AFS | | 5²⁰4⁵22²¹ |
| HG-AES | | 200¹⁸30¹⁸30¹⁸ |

^{a}This work.
^{b}Values correspond to the laser tuned off(on)-wavelength.

For completeness, the limits of detection obtained by HG-LIF using measurements of the blank on wavelength were also determined. Due to the limited purity of the reagents, the limits of detection are degraded somewhat when the laser is tuned on wavelength, typically by a factor of 5 for each element. Accordingly, the on wavelength LODs are 2 pg ml-1, 0.5 pg ml-1 and 1 pg ml-1 for As, Se and Sb, respectively. Additionally the concentration equivalent of the intercept of the regression line was calculated for each element. These values corresponded to −1.6 pg ml-1, 1.7 pg ml-1 and −0.2 pg ml-1 for As, Se and Sb, respectively. While these results suggest that the HG-LIF techniques are limited by the purity of reagents, they also indicate that the lower range of measurement of the approaches can be extended with higher purity reagents and is not inherently limited by the LIF detection approach.

### HG-LEI of Se and As

In the present Example, LEI detection of As was achieved by stepwise excitation at 197.197 nm followed by excitation at 435.531 nm from the 4p2 5s 4P1/2 level to the 4p2 7p 4Po3/2 level at 73647 cm-1. Two step LEI detection of Se was achieved by first step excitation at 196.026 nm followed by second step excitation at 433.028 nm from the 4p3 5s 3So1 to the 4p3 5p′ 3P2 level at 74083 cm-1. The measurement of As and Se using these schemes has been reported previously for batch sampling HG-LEI.25

Using continuous flow HG, the LEI signals were linear over a range of more than two orders of magnitude from 0.05 ng ml-1 to 10 ng ml-1 for both elements. The limits of detection for As and Se using HG-LEI were 50 pg ml-1 and 2 pg ml-1, respectively (Table 4). In order to account for small analyte background signals, the limits of detection were estimated using the standard deviation of 16 measurements of the blank with the UV laser detuned away from the resonance absorption line of the element. By comparison the on wavelength LODs were 100 pg ml-1 and 6 pg ml-1 for As and Se, respectively. Although a complete investigation of the limiting noises was not conducted, it appeared in these studies (as in previous studies using this apparatus25) that the limiting noise was due to sources other than RF pickup, i.e. flame ionization (shot and flicker noises).

Despite the inherently high signal collection efficiency possible in LEI approaches it appears that the lower sensitivity observed for HG-LEI compared to HG-LIF was due to an inability to saturate the LEI excitation transitions. This was verified through power dependence studies of the HG-LEI signals for As and Se in which the intensity of one laser was varied while the other was held constant. In these studies, saturation for As was achieved at about 2 μJ/pulse for the first step, but was unable to be achieved for the second step despite using up to 250 μJ/pulse. Similarly, saturation was achieved for Se at about 1 μJ/pulse for the first step and about 150 μJ for the second step. The relative magnitudes of these saturation energies suggest that nonradiative decay rates are much higher in the uppermost energy levels as compared to the intermediate levels, which prevents full saturation of the second step transition. The inability to saturate suggests that higher sensitivities are attainable with higher intensity laser excitation.

In comparison, to comparable HG-LIF approaches, HG-LEI measurements for As and Se are more complex (requiring two lasers) and do not provide as high sensitivity. Without more efficient laser excitation and an improved atomizer, it is not likely that HG-LEI will provide any significant advantages and will not be as useful for sub-ppb measurements of these elements.

### HG-ETA-LIF

Using an electrothermal atomizer (ETA) for in situ trapping and atomization can enhance HG measurement capabilities. For this reason, the utility of HG with in situ trapping and ETA-LIF detection was explored. The same LIF schemes used for HG-LIF in flame atomizers were also used for HG-ETA-LIF of As and Se. The HG-ETA-LIF approaches provided linear response to analyte concentrations to at least 1 ng ml-1 for As and Se. Measurements of higher concentrations were not performed in order to prevent contamination of the ETA and HG apparatus. Absolute mass LODs of 6 pg and 40 pg were obtained for As and Se, respectively, and correspond to concentration LODs of 3 and 20 pg ml-1. These results are comparable to those reported for HG-AAS and HG-ICP-MS trapping approaches in the ETA (Table 5). The limits of detection were estimated using the standard deviation of 16 measurements of the blank with the laser tuned onto the resonance absorption line of the element.

TABLE 5
LODs for several HG-atomic spectrometry techniques that utilize
in situ trapping in electrothermal atomizers

| | |
|---|---|
| | LOD/pg ml⁻¹ (pg) |
| | As |
| | Se |
| HG-ETA-LIF^{a} | 3 (6)^{b}20 (40)^{b} |
| HG-ETAAS | 20 (17.5 ± 1)¹³ 20 (22.5 ± 1)¹³ |
| | 13 (92)¹⁶17 (120)¹⁶ |
| | 4.3 (43)¹⁴60 (60)¹⁷ |
| | 0.8 (8)¹⁴ |
| HG-ICP-MS | 0.6 (2.9)¹²396 (1980)¹² |

^{a}This work.
^{b}Values correspond to the laser tuned on-wavelength only.

It is noteworthy that the ETA-LIF technique itself is able to achieve absolute mass LODs of 200 fg for As and Se,24 which are approximately 1 and 2 orders of magnitude better than that achieved using HG-ETA-LIF. The lower sensitivity observed for the HG-ETA-LIF approach suggests losses in the hydride transport and/or trapping processes, especially for Se. However, since virtual 100% transport/trapping efficiency has been reported previously for HG combined with ETAAS and ICP-MS detection, it is anticipated that higher sensitivity may yet be obtained by the HG-ETA-LIF approaches.

In comparison to HG-LIF in flame atomizers and HG-LEI, HG-ETA-LIF provides provides good sensitivity but is less reproducible and less suited to continuous measurements in an “on-line”format. In order to achieve the highest sensitivity, significant trapping of the evolved hydrides is required, which increases the measurement times. Also, the hydride trapping step and transient signal produced by the ETA both provide additional sources of variability into the measurements that are absent in the HG-LIF (flame) and HG-LEI approaches. Furthermore, the conventional backscatter signal collection in the ETA is much more prone to noises due to laser scatter and window fluorescence than are the other two approaches. Still, it may be that the combination of hydride trapping and ETA-LIF will be able to provide the absolute lowest detection limits in low concentration samples.

### Speciation of As and Se Using HG-LIF with Flame Atomization

The Examples demonstrate the speciation and quantitative recovery of As and Se by the flame HG-LIF approaches. Measurements of As (V) and As (III) were performed in two different solution systems, 4% HCl and 2.5 M Tris-HCl buffer solution. In 4% HCl, both As(V) and As(III) are reduced to arsine (AsH3), while only As(III) is reduced to AsH3 in the 2.5 M Tris-HCl. The LODs for As were identical in both solution systems. Calibration plots were prepared for both sets of measurements. Regression parameters, including the standard deviations of the residuals (sy), slopes and intercepts obtained for each set of HG conditions are shown in Table 6. Since the solutions in each set contain equal amounts of As(III) and As(V), the slope observed in the HCl solution was expected to be twice that observed in the buffered solution. The results given in Table 6 indicate that speciation (selective HG) and quantitative recovery of the two As species were achieved. In addition, high precision was also demonstrated by the low standard deviations obtained for the slope, y-intercept and residuals.

The same measurements were performed for Se using solutions containing equal concentrations of Se(IV) and Se(VI) species. In one approach, the solutions were measured directly without prereduction, in which case only the Se(IV) species produced a signal. In the other approach, the samples were subjected to a prereduction step (boiled at 100° C.) to convert the Se(VI) species to the Se(IV) species. Subsequent HG resulted in a measurement of total inorganic Se (Se(VI)+Se(IV)). The LODs of the HG approaches were identical for both Se species. Regression parameters from the calibration plots for both HG conditions, including the standard deviation of the residuals, slope and intercept, are given in Table 6. Since the solutions each contain equal concentrations of the two Se species, the two approaches were expected to provide slopes differing by a factor of two. The results indicate that speciation (selective HG) and quantitative recovery of the two Se species were achieved. The HG approaches also provided high precision as demonstrated by the small standard deviations obtained for the slope, y-intercept and residuals.

TABLE 6
Regression parameters obtained for selective HG studies of As and Se^{a}

| | m ± sₘ/ | | |
|---|---|---|---|
| Selectivity conditions | (a.u.) ml ng⁻¹ | b ± s_{b} (a.u.) | s_{y} (a.u.) |
| As(III + V), pH = 6.2 | 2.22 ± 0.01 | 0.00 ± 0.01 | 0.01 |
| As(III + V), pH 0-1 | 4.65 ± 0.06 | 0.02 ± 0.03 | 0.05 |
| Se(IV + VI), room temperature | 34.6 ± 0.5 | 0.1 ± 0.2 | 0.30 |
| Se(IV + VI), 100 ° C. | 67.4 ± 0.9 | 0.0 ± 0.4 | 0.49 |

^{a}a.u. = Arbitrary units.

### Conclusion

These Examples, effectively demonstrate the exceptionally high sensitivity that can be achieved from combining continuous flow HG with LIF detection in a simple H2/air diffusion flame atomizer. The sensitivities and sub-pg ml-1 LODs reported here for the flame HG-LIF techniques are in all cases equal to or better than those reported for measurements of As, Se and Sb by any HG approach, including HG-ICP-MS.

In addition to high sensitivity, the LIF techniques possess excellent spectral selectivity and are not susceptible to the same spectral or polyatomic interferences that affect AAS or ICP-MS measurements of these elements, e.g. 75As (40Ar35Cl) and 80Se (40Ar40Ar). The high spectral selectivity provided by LIF is a major advantage and is crucially important for maintaining accuracy at low pg ml-1 concentrations.

The combination of selective HG procedures with LIF detection in the flame atomizer has also been investigated. As a result of the high sensitivity of LIF detection, it has been shown that the major As and Se oxyanions can be selectively and quanitatively measured at the sub-ppb and pptr levels. It is worth noting that with the advances already made in solid state tunable laser technology, the exceptional level of performance of the flame HG-LIF techniques reported here can be implemented and operated conveniently and with less cost than that of an ICP-MS system.

In addition to flame HG-LIF techniques, the capabilities of continuous flow HG with ETA-LIF detection and LEI detection have also been investigated for low level measurements of As and Se. Although neither approach is currently as sensitive as the corresponding flame HG-LIF approach, the HG-ETA-LIF approaches appear to have the best potential for improvement in terms of sensitivity, especially with efficient trapping of accumulated hydrides in the ETA prior to LIF measurements.

As a further embodiment of the present invention, it is envisioned that ion chromatography (IC) approaches be combined with HG-LIF detection for on-line speciation of As and Se compounds. Among the attractive features of IC are its relatively short analysis times, variable sample volume requirements (ml-ml) and capability of simultaneously analyzing more than one As or Se compound, including the alkylated forms As and Se species with applications to environmental and biological samples.

While the illustrative embodiments of the invention have been described with particularity, it will be understood that various other modifications will be apparent to and can be readily made by those skilled in the art without departing from the spirit and scope of the invention. Accordingly, it is not intended that the scope of the claims appended hereto be limited to the examples and descriptions set forth herein but rather that the claims be construed as encompassing all the features of patentable novelty which reside in the present invention, including all features which would be treated as equivalents thereof by those skilled in the art to which this invention pertains.

We claim:

1. An apparatus for processing a sample comprising a compound of interest, the apparatus comprising:
(a) a hydride generator suitable for generating a hydride of the compound from the sample; and
(b) a laser-based detection device positioned to receive the hydride from the hydride generator, and to provide information regarding quantity, identity, presence and/or species of the hydride.

2. The apparatus of claim 1, wherein the compound comprises at least one selected from the group consisting of arsenic, selenium and antimony.

3. The apparatus of claim 1, wherein the laser-based detection device is a laser-induced fluorescence device.

4. The apparatus of claim 1, wherein the laser-based detection device is a laser-enhanced ionization device.

5. The apparatus of claim 1, wherein the hydride generator is a continuous flow hydride generator.

6. The apparatus of claim 5, wherein the laser-based detection device is a laser-induced fluorescence device.

7. The apparatus of claim 5, wherein the laser-based detection device is a laser-enhanced ionization device.

8. An apparatus for processing a sample comprising a compound of interest, the apparatus comprising:
(a) a hydride generator suitable for generating a hydride of the compound from the sample;
(b) an electrothermal atomizer positioned to receive and atomize the hydride from the hydride generator;
(c) a laser-based detection device positioned to receive the atomized hydride from the atomizer, and to provide information regarding quantity, identity, presence and/or species of the hydride.

9. The apparatus of claim 8, wherein the compound comprises at least one selected from the group consisting of arsenic, selenium and antimony.

10. The apparatus of claim 8, wherein the laser-based detection device is a laser-induced fluorescence device.

11. The apparatus of claim 8, wherein the laser-based detection device is a laser-enhanced ionization device.

12. The apparatus of claim 8, wherein the hydride generator is a continuous flow hydride generator.

13. The apparatus of claim 12, wherein the laser-based detection device is a laser-induced fluorescence device.

14. The apparatus of claim 12, wherein the laser-based detection device is a laser-enhanced ionization device.

15. A method for processing a sample, comprising:
(a) generating a hydride from the sample; and
(b) generating information regarding quantity, identity, presence and/or species of the hydride.

16. The method of claim 15, wherein step (a) is carried out utilizing a continuous flow hydride generator.

17. The method of claim 15, wherein step (b) is carried out utilizing a laser-based device.

18. The method of claim 17, wherein the laser-based device is a laser-induced fluorescence device.

19. The method of claim 17, wherein the laser-based device is a laser-enhanced ionization device.

20. The method of claim 15, wherein step (a) comprises generating at least one hydride selected from the group consisting of hydrides of arsenic, selenium and antimony.

21. A method for processing a sample, comprising:
(a) generating a hydride from the sample;
(b) atomizing the hydride and
(c) generating information regarding quantity, identity, presence and/or species of the hydride.

22. The method of claim 21, wherein step (a) is carried out utilizing a continuous flow hydride generator.

23. The method of claim 21, wherein step (c) is carried out utilizing a laser-based device.

24. The method of claim 23, wherein the laser-based device is a laser-induced fluorescence device.

25. The method of claim 23, wherein the laser-based device is a laser-enhanced ionization device.

26. The method of claim 21, wherein step (a) comprises generating at least one hydride selected from the group consisting of hydrides of arsenic, selenium and antimony.
